(19) United States
(12) Patent Application Publication
(10) Pub. No.: US 2026/0188440 A1
(43) Pub. Date: Jul. 2, 2026

(54) Reverse Data Exchange Mapping and Integration

(71) Applicant: Akyrian Systems, Inc., New York, NY (US)

(72) Inventors: Edward Ikeguchi, New York, NY (US); Gordon Kessler, Mount Kisco, NY (US)

(21) Appl. No.: 19/178,460

(22) Filed: Apr. 14, 2025

Related U.S. Application Data
(60) Provisional application No. 63/641,065, filed on May 1, 2024.

(51) Int. Cl. G16H 10/20; G16H 10/60

(52) U.S. Cl. CPC G16H 10/20; G16H 10/60

(57) ABSTRACT

A system and method for reverse data exchange mapping are provided. The method includes receiving information for identifying a group of computerized records, receiving an indication of a sub-grouping of records, and receiving one or more input entry values identified by a user. The input entry value is matched with one or more record values in the sub-grouping of records, and a link between the input entry value and the record value is confirmed.

### CROSS-REFERENCE TO RELATED APPLICATIONS

This application claims the benefit of U.S. Provisional Patent Application Ser. No. 63/641,065 to Edward Ikeguchi, filed May 1, 2024, titled “Reverse Data Exchange Mapping and Integration”, currently pending, the contents thereof being incorporated herein by reference.

### BACKGROUND

Implementation of data exchange systems has been difficult to achieve. A typical mapping of data fields during a system integration to transfer data from a source system to a receiving system requires a pre-determination of every field that might be necessary, and then a pre-determination of one or more codes from the system from which the data is stored so that the system to which the data is to be transferred can properly classify the information. This results in a system that must be customized for each use, and indeed, if an additional bit of information is to be transferred, the code of the system must be rewritten. Moreover, in a typical Application Programming Interface (API), the receiving system must request information from a source system, identifying a field corresponding to the desired information. This requires the receiving system to know for which information it is asking.

This data exchange problem is particularly acute in the healthcare field. The technology underlying electronic health record (EHR) keeping has made tremendous strides in creating a healthcare data ecosystem that is more readily accessible to patients, providers, and a host of administrative users. In the United States, for example, the vast majority of hospitals and clinics have moved from using paper medical charts to EHR systems. The arrival of EHR systems has also come with the need to create standards for data storage and database structure such that disparate systems created by multiple vendors can communicate with each other. For example, a hospital with an EHR system might need to communicate with dozens of other electronic systems to gather information for a patient about their lab tests, radiology, pathology, consultations with other physicians, results from other hospitals, surgical reports, etc.

In order to facilitate the communication of health information systems, various frameworks have been created to serve as standards for structuring the exchange of electronic healthcare data. One such framework is Health Level-7 (HL-7) with an application programming interface (API) known as Fast Healthcare Interoperability Resources (FHIR). Such frameworks have been broadly adopted to support data standardization to facilitate smooth transmission of information and interoperability between healthcare database systems.

One obvious benefit of the migration of almost all hospitals and clinics towards electronic recordkeeping would be the massive pool of patient information that could be used by drug makers to support the clinical trials needed to research new therapeutics. However, even after a decade of available healthcare informatics standards, there is little if any adoption of methods deploying data acquisition directly from EHR systems vis-a-vis APIs such as FHIR.

Therefore it would be beneficial to provide an improved system that would facilitate the adoption of methods deploying data acquisition directly from EHR systems vis-a-vis APIs such as FHIR.

### SUMMARY

The inventors of the present invention have recognized that the failure of adoption of methods deploying data acquisition directly from EHR systems vis-a-vis APIs such as FHIR is not for a lack of trying. Rather, this is due to the fact that there is heterogeneity between manufacturers of EHR systems, where database structures and nomenclature are only partially harmonized. In fact, even amongst doctors at the same hospital, there may be idiosyncrasies in database structure within the same EHR system. Furthermore, a receiving database provisioned by a research sponsor needs to utilize an API such as FHIR to request a specific data field from the source (hospital) system in order to inform the API what to fetch. This request must accompany a means of uniquely identifying the desired data field in the source system. While conventions or standards around unique identifiers in healthcare data exist, however, with most of these conventions there are multiple possible unique identifiers for a single clinical observation. A clinical observation is herein defined as a parameter that is descriptive of the human body (such as a vital sign, a physical examination parameter, or a parameter determined by a laboratory test), that is articulated in data that can be stored in a data field or a variable within a database. A data field, data point, datapoint, or variable is herein defined as a discrete storage unit within a database that can be identified vis-à-vis a unique identifier. The unique identifier is typically a numeric designation or code. There are hundreds of thousands of observations and many multiples of variations on a single observation. Thus the conventions used to uniquely identify data fields are complicated by multiple options from which to choose when trying to deploy a unique identifier. For example, a simple observation such as “Temperature” might have hundreds of possible unique identifiers for variations on the one observation (“Basal Temperature”, Axillary Temperature”, etc.). In actuality, the only stakeholder who has definitive knowledge of what is meant by a particular observation in an EHR is the clinician themselves. The problem is further compounded by the fact that the people developing and configuring the receiving database on behalf of the clinical trial research sponsor are not clinical personnel providing care to patients

This problem is directly applicable, for example, to data acquisition and record keeping in clinical trials. While most large pharmaceutical companies have attempted to deploy pilot clinical trials as proof-of-concept projects, several factors remain as barriers to mainstream usage. Attempts at sharing data between hospital systems and clinical research systems built for data collection (such as electronic data capture) have struggled. The inventors of the present invention therefore present a system and method that would in part allow the clinician to choose the correct unique identifier to be used in a FHIR API request for a particular observation.

A “Doctor” or “Clinician” is defined herein as a professional who serves as a provider of healthcare products and services. The Doctor or Clinician who acts as a researcher in a clinical trial is also referred to as an “Investigator.” The investigator's hospital or clinic where they perform clinical trial activities is referred to as an “Investigator Site” or “Site.” A patient is referred to as a “Study Subject” or “Subject” if they are participating in a clinical trial. Study Subjects and Investigators meet at pre-defined intervals for ongoing treatments and wellness evaluations which are referred to as a “Visit” or “Visits”. Investigators and other site personnel are presented with questionnaires from pharmaceutical companies performing research on novel medical treatments. These questionnaires are referred to as “Case Report Forms” (“CRF”, “CRFs”) and may be available in electronic form (“eCRF”, “eCRFs”). When CRFs are electronic, they are often presented in software packages known as “Electronic Data Capture” or “EDC.”

Problem 1: Every hospital or clinic is slightly different in their database implementation, making it difficult for pharmaceutical databases to interoperate with multiple different doctors (clinical investigators) and their offices (investigator sites or sites) without considerable bespoke development which entails tedious mapping of pharmaceutical research questions to variables within a hospital database. Note that the variables within the hospital databases are also organized by standards. For example, a frequently used coding system is Logical Observation Identifiers Names and Codes (LOINC) where clinical observations or lab tests can be categorized into logical groupings with unique numeric identifiers. However, these systems are massive in size, with multiple possible choices for a single patient parameter or observation. This makes the configuration of pharmaceutical data collections systems extremely difficult to accomplish before the start of a clinical trial. Furthermore, considering that a clinical trial may employ tens to hundreds of investigator sites, this leads to major challenges with scalability from a time and cost perspective.

Problem 2: While there are data exchange standards, there is a lack of controls in semantics. For example, while one doctor might refer to a patient's breathing troubles as “shortness of breath,” another doctor might document the same finding in their EMR as “dyspnea.” In an EHR, this ambiguity would be further exacerbated by a sentence like, “The patient experienced shortness of breath and chest pain during sex at home, which worsened upon arrival in the emergency department.” What became worse? The shortness of breath, the chest pain, or the sex? It is important to note that only the clinician understands the meaning of what they meant to say when documenting a patient encounter. For this reason, even with the availability of standards in semantics, there will always be a tendency towards variability when interpreting what a doctor documented. Also, for this reason, it is seen as an impractical task to custom integrate a particular hospital or clinic's EHR system for a particular clinical trial investigator (clinician) for any given question on a clinical trial questionnaire.

This issue is well supported in the scholarly literature, as recently described in the Journal of Medical Internet Research by Amar et al. in the 2024 manuscript entitled, “Electronic Health Record and Semantic Issues Using Fast Healthcare Interoperability Resources: Systematic Mapping Review.” In it the authors conclude, “Semantic interoperability is still an active research field, and there is no broad consensus in the health care community on how to achieve full semantic interoperability between information systems. The main issues reported are related to the difficulty associated with the use of different terminologies and the efforts required to successfully map to FHIR terminology. Even worse, when no terminology is used, unstructured data would still need to be extracted and mapped to the appropriate concept code for FHIR terminology.”

The present invention solves both of the current issues (Problem 1 and Problem 2) by removing the need for a pharmaceutical company or other entity to map a question from a clinical trial database to a hospital system prior to launching the project. Furthermore, the inventive system and method achieve the integration at a datapoint level that is completely agnostic to which brand of EHR is utilized by the investigator at their hospital or clinic. Lastly, the inventive system and method puts the process of data mapping and semantics mapping into the hands of the clinician who is most intimately familiar with the intended meaning of a particular value entered into an EHR system and how it relates to a clinical trial question.

In accordance with one or more embodiments, a reverse data exchange mapping system is provided. Training of the receiving system is performed at the same time as use. A user can determine a source system from which data will be requested. Once a link is established, all data from a particular record of the source system may be transferred to the receiving system. Instead of needing to know the field codes (i.e. LOINC number) ahead of time, the receiving system instead searches the data comprising observations entered by a user, typically the clinician (i.e. Investigator) or their designee at a hospital or clinic, among the data received from the source system. Once a clinician confirms that a match is found, it is then possible to identify the correct unique identifier and link it to the field in the source system corresponding to the observation requested by the eCRF in the receiving system. Once the correct link is made it is possible to automate the current and future transfer of the indicated data from the source system (EHR) to a particular field in the receiving system (eCRF) so all future data may be automatically transferred between these fields systems and inserted into the proper fields. If more than one possible data value is determined to be present in the source system data, the user may preferably be given an opportunity to manually select which fields are to be linked.

In this manner, data field links are determined during use and do not require any pre-determined knowledge of field codes or the like. An exemplary embodiment of the invention may employ a FHIR (Fast Healthcare Interoperability Resources) Integration of healthcare data. FHIR is a healthcare data exchange standard that leverages data resources (XML, JSON) and RESTful architecture to power seamless real-time data exchange. Details of the implementation of an embodiment of the invention to the FHIR data transfer protocol will be described below.

### BRIEF DESCRIPTION OF DRAWINGS

The invention will be described making reference to the drawings, in which:

FIG. 1 is a flowchart outlining steps in accordance with an embodiment of the invention;

FIG. 2a is a first portion of a flowchart outlining steps in accordance with an embodiment of the invention as applied to an integration employing reverse mapping;

FIG. 2b is a second portion of a flowchart outlining steps in accordance with an embodiment of the invention as applied to an integration employing reverse mapping;

FIG. 3a is a screen shot depicting functionality in accordance with an embodiment of the invention as applied to an integration employing reverse mapping; and

FIG. 3b is a further screen shot depicting functionality in accordance with an embodiment of the invention as applied to an integration employing reverse mapping.

### DETAILED DESCRIPTION

In accordance with various embodiments of the invention, the term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program include, by way of example, can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer, mobile, or other electronic device designed to receive information input by a user.. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

While this specification contains many specific implementation details, these should not be construed as limitations on the scope of any subject matter described in this disclosure or on the scope of what may be claimed, but rather as descriptions of features that may be specific to particular embodiments of the subject matter described in this disclosure. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

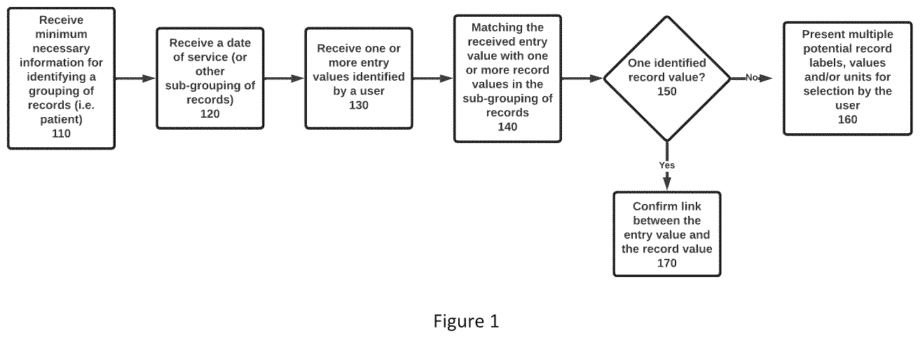

Referring first to FIG. 1, a description of a Reverse Data Exchange Mapping and Integration method and system constructed in accordance with an embodiment of the invention is depicted. At step 110, a minimum amount of information necessary for identifying a grouping of records in the source database is received. Such grouping may be the medical observation data of a single patient (i.e. values reported in an electronic medical record related to a patient, such as vital signs, test results, etc.), reports from an access log for a particular location, machine malfunction logs, or the like. Processing then passes to step 120 where a further input allows for designation of a particular sub-grouping of the grouping of records. Such sub-grouping may be a date of service, location of one or more machines, etc. Thereafter, processing continues at step 130 where one or more entry values are received from a user or other input mechanism, such as an AI generated input, database readout, or the like.

Then, at step 140, the one or more entry values received from the user at step 130 are attempted to be matched with values present in the identified sub-grouping of records. For example, if a heart rate number is being searched for, the user might enter “98”, and thereafter, at step 150, the system will indicate whether there is a match to this value in any of the records in the sub-grouping. If multiple records are retrieved that include this value, they may all be presented to the user, preferably along with record labels (identifying the name of the observation (i.e. “temperature” or “heart rate”) and/or units (i.e. “degrees Fahrenheit” or “beats per minute”)) to aid the user in selecting the correct record, at step 160. The user may then select one of the records as the desired record to be linked. Alternatively, at step 150, it may be determined that there is only one record that includes the indicated value. Processing then continues either from step 150 or 160 to step 170 where a link is confirmed between the entry value and the record value.

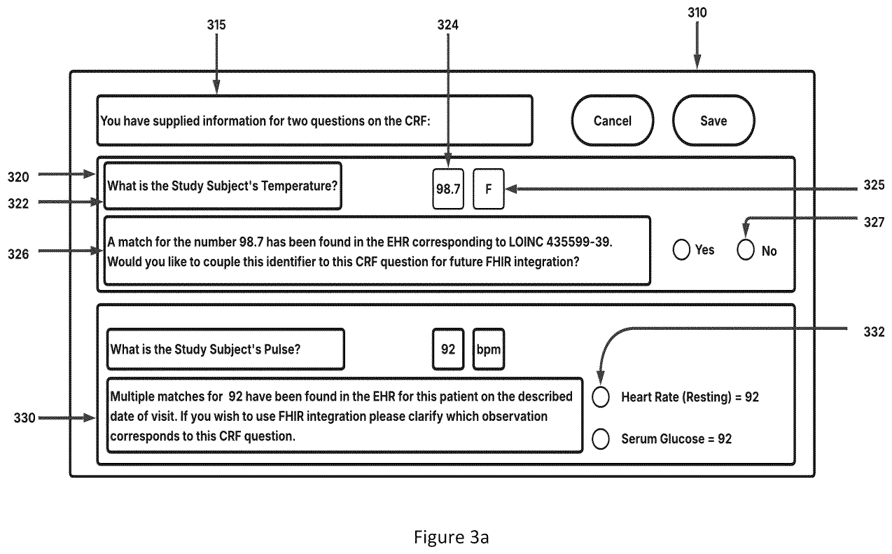

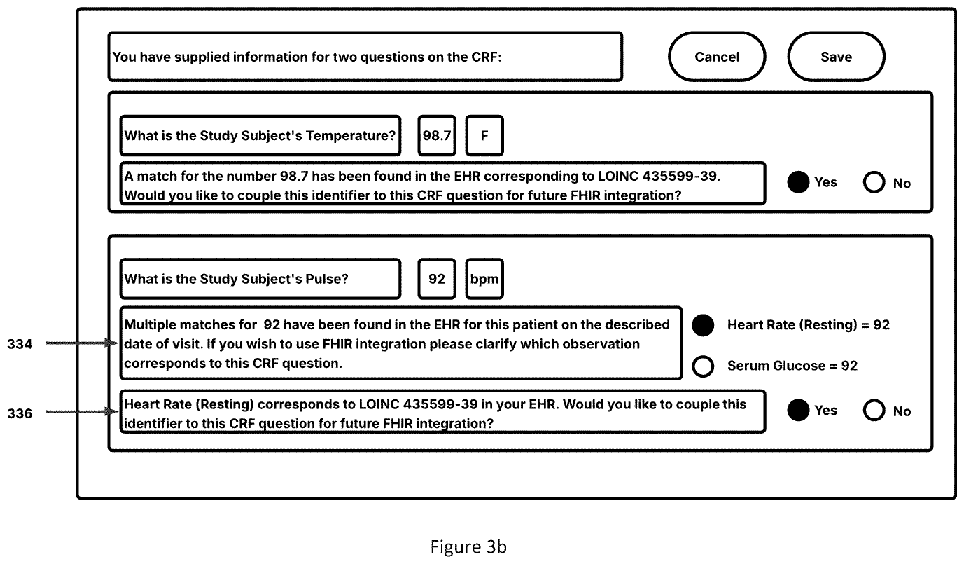

Referring next to FIGS. 3a and 3b, the system as presented to a user and interaction between the system and a user will be described. A user is presented with a screen 310 on a computing device. At 315 the user is shown text indicating the information that has been supplied for answering questions in the CRF. In this case, the user has presented information to answer two such questions. The user is then shown 320 indicating that the first question in the CRF is related to the study subject's temperature and 330 indicating that the second question in the CRF is related to the study subject's pulse. At 324 the user then enters the temperature number (98.7) for the study subject, and at 325 the user enters the unit (F) for the temperature. Then at 326 the user is notified that a match for the number 98.7 has been found in the EHR corresponding to a LOINC code (LOINC 435599-39) and is asked whether they would like to couple this identifier to this CRF question for future FHIR integration. At 327 the user can confirm (Yes) or reject (No) the coupling of the CRF question to the LOINC identifier. Once confirming Yes, there is a permanent link formed and future requests for temperature in the CRF can be automatically transferred from the EHR. For the second question shown at 330 to document a study subject's pulse, the same initial process is employed. In this case, however, at 330 the user is notified that multiple matches for 92 (the entered pulse rate) have been found in the EHR for this patient on the described date of visit. The user is therefore notified that if they wish to use FHIR integration they will need to clarify which observation corresponds to this CRF question at 332 (either “Heart Rate (Resting)” or “Serum Glucose”. In FIG. 3b, once “Heart Rate (Resting)” has been selected at 334, then the user is notified at 336 (similar to 330) that the Heart Rate (Resting) corresponds to LOINC 435599-39 in the EHR and is asked whether they would like to couple this identifier to this CRF question for future FHIR integration. Similarly to 332, the user can confirm (Yes) or reject (No) the coupling of the CRF question to the LOINC identifier. Once confirming Yes, there is a permanent link formed and future requests for temperature in the CRF can be automatically gleaned from the EHR.

Once confirmed, the entry value may then be correlated with the record type (via unique identifier) so that in the future, when the particular type of value is desired to be automatically input, the value can be retrieved from the linked record. In addition, or as an alternative, to receiving an entry value, the user may provide an entry unit of measure. In such a situation where only the unit of measure is provided, rather than simply searching for a particular value, the system may instead search for one or more fields in the sub-grouping that are defined as including this unit of measure. If both an entry value and an entry unit of measure are provided, searching and filtering for these two entry values may help to locate and/or focus in on a single particular field. For example, as noted above, there may be more than one field matching a particular value (i.e. a “8” may be a valid entry for heart rate, glucose, and temperature). By also referring to the entry unit of measure, the system can differentiate between these fields with identical entry values.

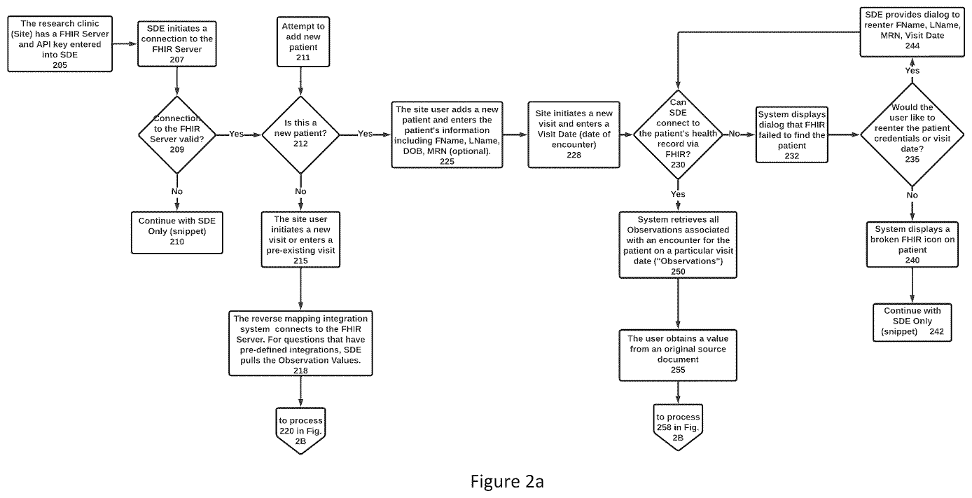

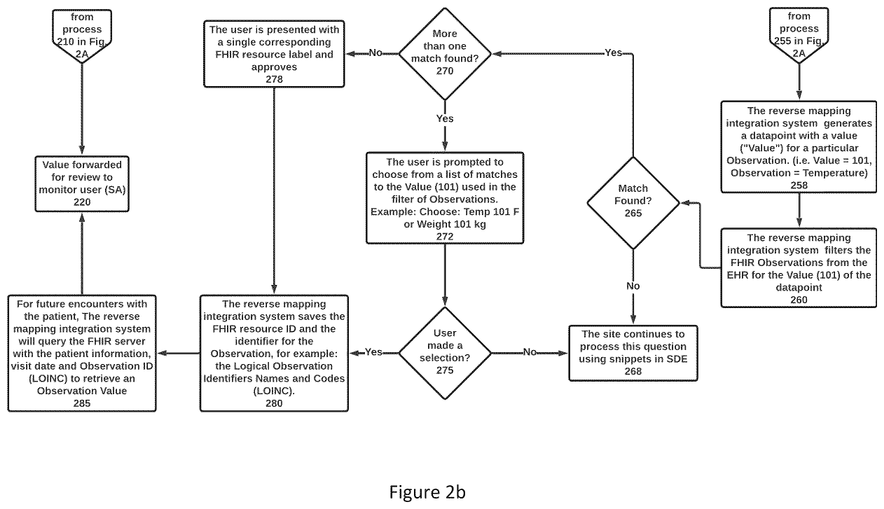

Referring next to FIG. 2, a more particular example of an embodiment of the invention will be described. FIG. 2 depicts application to implementation of a FHIR integration employing reverse mapping for entering Electronic Heath Record (EHR) data into a database designed to collect clinical trial data in an electronic Case Report Form (eCRF), such as an Electronic Data Capture system, or in an additional embodiment, a Source Data Extraction (SDE) system of the type disclosed in the following patents and applications sharing inventorship with the inventor of the present invention, the entire contents of each of these patents and applications being incorporated herein by reference: U.S. Pat. No. 10,706,958; U.S. Pat. No. 10,811,122; U.S. Pat. No. 11,562,811; U.S. Pat. No. 11,562,810; U.S. patent application Ser. No. 18/084,788; U.S. patent application Ser. No. 18/084,969; and U.S. patent application Ser. No. 18/534,142. Of course, the inventive solution presented herein may be employed in any mapping situation where a user is able to provide an entry value and/or an entry unit of measure to thus search a source database. The user is able to discover a unique identifier of a field in the source database that can in the future be saved and referenced by the receiving database to automate the transfer of information from the source database to the receiving database. Thus, a unique field is defined in the source database to which a corresponding field in a receiving database is to be mapped.

As is shown in FIG. 2, processing begins at step 205 where a user from an investigator site enters a FHIR Server location and API key into a reverse mapping integration system, such as an EDC system, or an SDE system of the type described above (the reference to FHIR, LOINC, and the like being exemplary only). At step 207 the reverse mapping integration system initiates a connection to the FHIR Server and at step 209 queries whether the connection to the FHIR Server is valid. If it is determined that the connection is not valid, processing continues at step 210 where the FHIR integration cannot be performed, and the Site therefore continues employing an alternative or more traditional data entry procedure, such as a snippet-based data entry procedure when employing an SDE system of the type described above.

If on the other hand it is determined at step 209 that the connection to the FHIR server is valid, a user may attempt to add a study subject entry to the inventive system at step 211, and at step 212 it is queried whether this entry is for a new study subject. If the query at step 212 determines that study subject entry is not for a new study subject then processing continues at step 215 where the user from the investigator site initiates data entry for a new visit or a pre-existing visit for the existing study subject. Because the reverse FHIR mapping process will already have taken place with a pre-existing study subject, the reverse mapping integration system is able to pull observation values from any questions that have pre-defined integrations, effectively automatically answering questions in a clinical trial eCRF by pulling or fetching values from predefined fields from a medical record in the EHR system at step 218. Processing then completes at step 220 where the obtained observation value is presented to the user from the investigator site for review and confirmation.

If on the other hand it is determined at step 212 that the entered study subject is a new study subject, processing passes to step 225 where the site user adds a new study subject and enters one or more of the study subject's information preferably including First Name (FName), Last Name (LName), Date of Birth (DOB), and/or Medical Record Number (MRN). Then at step 228 the site user preferably initiates a new visit in the inventive system and enters a visit date (date of the encounter) for the new patient. At step 230 it is then queried whether the reverse mapping integration system can connect to the study subject's EHR via FHIR. If it is determined at step 230 that the reverse mapping integration system cannot connect to the study subject's health record via FHIR, processing continues with step 232 and displays that the FHIR system failed to find or connect to the new study subject. At step 235 the user at the investigator site is then given the opportunity to re-enter study subject credentials or the visit date. If the credentials or date are re-entered, these new values are passed back to the system, and processing returns to step 230. If on the other hand the user at the investigator site does not re-enter the credentials or visit date, processing passes to step 240 where an indication is provided to the user that the study subject data cannot be retrieved using the FHIR system, and at step 242 the site therefore continues employing an alternative or more traditional data entry procedure, such as a snippet-based data entry procedure when employing an SDE system of the type described in one or more of the above referenced applications incorporated herein.

If at step 230 it is instead determined that the reverse mapping integration system can connect to the study subject's health record via FHIR, processing continues with step 250 where the reverse mapping integration system retrieves all observations associated with an encounter for the study subject on a particular visit date from the EHR (as provided in step 228) (“Observations”). In the event the investigator site user is using SDE, the SDE system then obtains a source capture from the EHR and extracts a value for an observation from the original source document and may include a complete or redacted version of a source capture such as those defined in one or more of the above patents or patent applications incorporated herein by reference at step 255. Alternatively, redaction may be skipped, and the identifiable information may be retained by the system or redacted automatically or manually at a later time. In the event the investigator site user is using an EDC system (and therefore cannot benefit from the automated data transmission using the SDE system), the user views the information in the EHR and manually transcribes the value for an observation from the original source document in the EHR to the EDC system. Processing then continues at step 258 where the reverse mapping integration system generates a datapoint with a value (“Value”) for a particular Observation from the redacted source capture. (i.e. Value=101, Observation=Temperature). (Units of measure may be used alternatively or in addition to the Value, as described above.) When employing an SDE system as described above, such a datapoint may be referred to as a snippet from an original source document. Then at step 260 the reverse mapping integration system filters the retrieved FHIR observations for the value (i.e. “101”) associated with the datapoint (and/or unit of measure, if provided).

Processing continues at step 265 where it is queried whether a match is found between the value and/or unit of measure, if provided, associated with the datapoint retrieved from the SDE or EDC system and one or more of the values and/or unit of measure, if provided, associated with the one or more observations retrieved from the EHR system at step 250. If no match is found, processing continues at step 268 where the site user therefore continues employing an alternative or more traditional data entry procedure, such as a snippet-based data entry procedure when employing an SDE system of the type described above. If it is instead determined at step 265 that one or more matches are found, a second query at step 270 asks whether more than one match was found. If only one match was found, processing continues at step 278 where the site user is presented with a single FHIR resource label or LOINC code corresponding to the datapoint in the EHR whose value corresponds with the value obtained vis-à-vis the site user using SDE or EDC to approve. If more than one match is found, the processing from step 270 proceeds to step 272 where the site user is prompted to choose from a list of matches to the Value (i.e. “101”) found in the EHR for the sub-grouping of records for a particular patient, visit and observation. Example: Choose: Temp 101 F or Weight 101 kg. It is then confirmed whether the user makes a selection at step 275. If not, processing continues at step 268 and the site therefore continues employing an alternative or more traditional data entry procedure, such as a snippet-based data entry procedure when employing an SDE system of the type described above or a manual transcription of data from EHR to EDC.

Once the site user has made a selection and confirmed a relationship, whether from a group of selections at step 275 or from a single presented selection at step 278, processing then moves to step 280 where reverse mapping integration system saves a FHIR resource ID and the identifier for the Observation, for example: the Logical Observation Identifiers Names and Codes (LOINC). This generates a permanent link between the eCRF field and the particular observation in the EHR (or other applicable system pairs). Based upon this link, as noted at step 285, for future encounters with the study subject, the reverse mapping integration system will query the EHR via the FHIR API with the study subject information and visit date to retrieve a value from a field in the EHR with the corresponding LOINC for entry into the eCRF in an automated fashion. This retrieval may be employed for all potential observation values for a particular study subject and visit date, thus pre-populating a form to be filled in the SDE system automatically from EHR records via the FHIR integration. After retrieval, processing concludes at step 220 where the retrieved value is forwarded to another user, typically a user who is external to the site who is tasked with quality controls, for confirmation of entry into the reverse mapping integration system form. An audit trail together with an ongoing history of matchings between LOINC assignments of observations in a hospital (source) database or EHR system to CRF questions in a receiving database can be maintained by the inventive system to apply machine learning to further facilitate accurate matchings and trend analysis across multiple different hospital systems. This can be used to support the use of a large language model (LLM) in future clinical trials.

Therefore, in accordance with the various embodiments of the invention, a reverse data exchange mapping can be set up for even the most complex data transfer and storage systems without having to pre-define any of the relationships that may be bespoke to particular users or institutions. Values matched between a system that stores source data and other systems that benefit from receiving this source data can be used to present potential appropriate data links for approval by a user, such as the investigator or other support staff such as a nurse or study coordinator in the context of a clinical trial (but which is equally applicable outside of the clinical trial or healthcare context), who would understand the originally intended meaning of an observation value as they entered it into the source data storage system, or for automatic approval. In the case of FHIR integration, the inventive system and method result in the ability to define links between an EHR system and an SDE or EDC system without the need for any pre-existing knowledge of the source database and the unique identifiers assigned to its variables, and seamlessly retrieving health record data from an EHR system and inputting that data into eCRF forms associated with a particular clinical trial. It is further contemplated that the inventive system and method may be used with any medical record data transfer between any data storage system and any other system where retrieval of the stored data on a regular basis would be valuable, including but not limited to patient medical record portals or apps, healthcare provider patient dashboards, and the like.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

It will thus be seen that the objects set forth above, among those made apparent from the preceding description, are efficiently attained and, because certain changes may be made in carrying out the above method and in the construction(s) set forth without departing from the spirit and scope of the invention, it is intended that all matter contained in the above description and shown in the accompanying drawings shall be interpreted as illustrative and not in a limiting sense.

It is also to be understood that this description is intended to cover all of the generic and specific features of the invention herein described and all statements of the scope of the invention which, as a matter of language, might be said to fall there between.

1. A method for reverse data exchange mapping, comprising:
receiving information for identifying a group of computerized records;
receiving an indication of a sub-grouping of records;
receiving one or more input entry values identified by a user;
matching the input entry value with one or more record values in the sub-grouping of records; and
confirming a link between the input entry value and the record value.

2. The method of claim 1, wherein the group of computerized records comprises entries reported in an electronic medical record related to a patient.

3. The method of claim 2, wherein the sub-grouping of records comprises a date of service of one or more of the computerized records related to the patient.

4. The method of claim 3, further comprising:
determining that the input entry value matches two or more of the one or more record values in the sub-grouping of records;
presenting to the user with the two or more matching record values along with corresponding record labels and units; and
receiving a selection input from the user indicating a correct match between the input entry value and one of the two or more matching record values.

5. The method of claim 4, wherein the input entry value is received based upon a field corresponding to an answer to a question in an electronic case report form associated with a clinical trial.

6. The method of claim 1, further comprising correlating a field corresponding to an answer to a question in an electronic case report form associated with a clinical trial with a record unique identifier in an electronic health record.

7. The method of claim 6, further comprising, when entering data into the electronic case report form for a patient visit on a subsequent date of service, automatically inputting a value into the field corresponding to the answer to the question in the electronic case report form associated with the clinical trial by retrieving the value from the field in the correlated electronic health record associated with the unique identifier.

8. The method of claim 7, wherein the unique identifier comprises a FHIR resource ID.

9. The method of claim 8, wherein the FHIR resource ID comprises a Logical Observation Identifiers Names and Codes (LOINC) code.

10. The method of claim 1, further comprising:
receiving one or more input entry units from a user;
matching the input entry unit with one or more record units in the sub-grouping of records; and
in the event there is more than one record value that matches the input entry value, identifying one or more of the matching record values that also have a matching unit.

11. A system for reverse data exchange mapping, comprising:
an electronic health record storing system for storing patient information;
an electronic case report for system for collecting data in a clinical trial;
a reverse data exchange module for:
receiving via a data input module information for identifying a group of computerized records stored in the electronic health record storing system;
receiving an indication of a sub-grouping of records of the identified group of computerized records;
receiving one or more input entry values identified by a user;
matching the input entry value with one or more record values in the sub-grouping of records; and
confirming a link between the input entry value and the record value.

12. The system of claim 11, wherein the sub-grouping of records comprises a date of service of one or more of the computerized records related to the patient.

13. The system of claim 12, wherein the reverse data exchange module further performs:
determining that the input entry value matches two or more of the one or more record values in the sub-grouping of records;
presenting to the user with the two or more matching record values along with corresponding record labels and units; and
receiving a selection input from the user indicating a correct match between the input entry value and one of the two or more matching record values.

14. The system of claim 13, wherein the input entry value is received based upon a field corresponding to an answer to a question in an electronic case report form associated with a clinical trial.

15. The system of claim 11, wherein the reverse data exchange module further performs: correlating a field corresponding to an answer to a question in the electronic case report form associated with a clinical trial with a record unique identifier in the electronic health record.

16. The system of claim 15, wherein the reverse data exchange module further performs: when entering data into the electronic case report form for a patient visit on a subsequent date of service, automatically inputting a value into the field corresponding to the answer to the question in the electronic case report form associated with the clinical trial by retrieving the value from the field in the correlated electronic health record associated with the unique identifier.

17. The system of claim 16, wherein the unique identifier comprises a FHIR resource ID.

18. The system of claim 17, wherein the FHIR resource ID comprises a Logical Observation Identifiers Names and Codes (LOINC) code.

19. The system of claim 11, wherein the reverse data exchange module further performs:
receiving via the data input module one or more input entry units from a user;
matching the input entry unit with one or more record units in the sub-grouping of records; and
in the event there is more than one record value that matches the input entry value, identifying one or more of the matching record values that also have a matching unit.
